(19) United States
(12) Patent Application Publication
(10) Pub. No.: US 2026/0049341 A1
(43) Pub. Date: Feb. 19, 2026

(54) METHOD TO GENERATE SUPERCOILED CIRCULAR DNA IN VITRO

(71) Applicant: UNIVERSITY OF MARYLAND, BALTIMORE COUNTY, Baltimore, MD (US)

(72) Inventors: Charles J. BIEBERICH, Baltimore, MD (US); Xiang LI, Baltimore, MD (US)

(21) Appl. No.: 19/367,288

(22) Filed: Oct. 23, 2025

Related U.S. Application Data
(63) Continuation of application No. PCT/US2024/026847, filed on Apr. 29, 2024.
(60) Provisional application No. 63/498,886, filed on Apr. 28, 2023.

(51) Int. Cl. C12P 19/34; C12N 9/00; C12N 9/90

(52) U.S. Cl. CPC C12P 19/34; C12N 9/90; C12N 9/93; C12Y 605/00

(57) ABSTRACT

Current minicircle production methods are slow, expensive, and difficult to perform under GMP conditions because, in most cases, the product is derived from bacteria. In contrast, HTLA-and CHTLA-based synthetic circular supercoiled DNA production can be done completely in a test tube, using chemically or enzymatically synthesized oligonucleotides, long single stranded DNA, and/or double stranded DNA.

### CROSS REFERENCE TO RELATED APPLICATIONS

This application is a continuation application claiming priority to International Patent Application No. PCT/US2024/026847 filed on Apr. 29, 2024, which claims priority to U.S. Provisional Ser. No. 63/498,886 filed on Apr. 28, 2023 in the name of Xiang LI and Charles J. BIEBERICH entitled “A METHOD TO PRODUCE SUPERCOILED CIRULAR DNA IN VITRO,” which is hereby incorporated by reference herein in its entirety.

### FIELD

The present invention relates to methods of generating supercoiled plasmid-vector less circular DNAs from two or more precursor DNA fragments such as single-stranded DNA, double-stranded DNA and/or oligonucleotides.

### BACKGROUND

Advances in our understanding of the molecular basis of disease has fueled the explosive growth of new gene-based treatments, also known as gene therapy. Gene therapy is the deliberate genetic modification of the DNA in patients'cells to achieve a specific therapeutic goal. Under development for three decades, this technology has matured in recent years and pharma has invested heavily in development of novel treatments. More than 800 U.S. clinical trials are currently in progress for a wide variety of single gene inherited disorders as well as acquired diseases including cancer and rheumatoid arthritis. To date, five gene therapies have received FDA approval: ZYNTEGLO®, to treat beta thalassemia; KYMRIAH™, a CAR T-cell therapy for relapsed or treatment resistant leukemia and lymphoma; LUXTURNA™ for inherited retinal disorder; SKYSONA® for cerebral adrenoleukodystrophy, and ZOLGENSMA® for spinal muscular atrophy. The global gene therapy market was valued at $9B USD in 2023 and is expected to reach $46B USD by 2030.

Historically, gene therapy relied heavily on viruses as gene delivery vehicles, and all five FDA-approved biologics are based either on lentiviruses or adeno-associated viruses. During production, therapeutic viruses are rendered incapable of replication, so that a productive viral infection does not ensue in the patient. While the approved virus-based gene therapies have good safety profiles, there are safety concerns that the therapeutic viruses can potentially recombine with other viruses during production or in patients, generating new viruses with unknown, and potentially harmful properties, including replication. Moreover, viruses have an inherent predilection for infecting specific cell types, severely limiting the range of diseases that can be targeted. This, and other concerns, including high production costs and laborious good manufacturing production (GMP) workflows, has driven the search for non-viral alternatives to genetically alter patients' cells. For example, naked DNA (i.e. not packaged inside a viral capsid) can be used to deliver therapeutic genes, however getting naked DNA into cells requires the use of lipid-based delivery systems, sonication, electroporation, or ballistic (i.e., gene gun) methods using DNA-coated gold particles. Alternatively, non-viral gene delivery methods have been developed based on circular DNAs called plasmids that are produced in bacteria. However, plasmids by necessity carry bacterial DNA sequences that elicit a patient immune response, limiting their effectiveness.

To address this problem, small circular DNAs termed “minicircles,” that are completely devoid of bacterial DNA have been developed. Minicircles contain only the therapeutic gene and DNA elements required to support expression of that gene; all or nearly all other DNA of bacterial origin, including antibiotic resistance genes, are removed, e.g., by an engineered recombination process that occurs in bacterial cells. Minicircles have been demonstrated to be a powerful gene delivery platform, outperforming plasmids and yielding higher and more durable therapeutic gene expression, however minicircles still have to be purified away from other bacterial components, including the original plasmid from which they are derived. The process is so complex that one production run can take upwards of 270 days. Enzymatic production of minicircle-like DNA in a test tube (i.e., Doggybone DNA) reduces production time to 50 days, but GMP production is still highly problematic.

There continues to be a need for improved methods that can scale production of circular DNA that is devoid of bacterial vector DNA (i.e. antibiotic resistance gene, origin or replication) in a GMP-friendly manner. A new technology with the potential to produce and scale, in a completely synthetic manner, circular supercoiled DNA is described herein. The technology uses a process termed “heteroduplex thermostable ligase assembly,” wherein, under specific conditions, single or double-stranded DNA precursors are denatured and annealed in the presence of a thermostable DNA ligase, with or without a thermostable type II topoisomerase, to create circular, predominantly supercoiled DNA molecules or “synthetic circular supercoiled DNA (^{SCS}DNA).

### SUMMARY

In one aspect, a method of forming Sticky-End Blocks (SEB) with 5′ or 3′ overhangs is described, the method comprising:
introducing at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature; and
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having 3′ overhangs.

In another aspect, a method of producing synthetic circular supercoiled DNA (^{SCS}DNA) is described, the method comprising:
introducing at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature; and
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced.

In still another aspect, a method of producing substantially supercoiled DNA is described, the method comprising:
introducing at least two precursor DNA fragments into a buffer medium comprising a thermostable DNA ligase enzyme and a thermostable type II topoisomerase, wherein the precursor DNA fragments will correctly assemble to generate a defined DNA sequence;
applying heat to a first temperature to cause the at least two precursor DNA fragments to denature;
lowering the temperature to a second temperature for: (i) annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and (ii) substantial supercoiling of the ^{SCS}DNA in the presence of the thermostable type II topoisomerase.

In another aspect, a method of producing substantially supercoiled DNA is described, the method comprising:
introducing the at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature;
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and
lowering the temperature to a third temperature and adding a bolus of type II topoisomerase to initiate substantial supercoiling of the ^{SCS}DNA in the presence of the type II topoisomerase.

In yet another aspect, a synthetic circular supercoiled DNA (^{SCS}DNA) is described, wherein the ^{SCS}DNA is devoid of any DNA of bacterial or viral origin.

Other aspects, features and embodiments of the invention will be more fully apparent from the ensuing disclosure and appended claims.

### BRIEF DESCRIPTION OF THE FIGURES

FIG. 1. Two-fragment HTLA generates supercoiled ^{SCS}DNA.

FIG. 2. ^{SCS}DNA produced by CHTLA is supercoiled. Two plasmid pMax-GFP ^{SCS}DNA precursors were prepared by digesting the plasmid with either KpnI or XhoI. The digested DNA was purified to remove restriction enzymes and mixed at a 1:1 ratio. ^{SCS}x-GFP ^{SCS}DNA was generated in a 10-cycle CHTLA reaction. Unreacted precursors were removed by treating the reaction mixture with T5 exonuclease. The ^{SCS}DNA was analyzed on an agarose gel without ethidium bromide. The gel was stained with ethidium bromide after electrophoresis was complete. P, plasmid pMax-GFP DNA prepared from bacteria; V, ^{SCS}DNA; L, 1-kb DNA ladder.

FIG. 3. CHTLA synthesis of a circular DNA using single-stranded oligonucleotides as precursors. Six overlapping 80-mer nucleotides overlapping by 40 bases were phosphorylated (by T4 polynucleotide kinase) at 37° C. and subjected to a CHTLA reaction with (left panel, lanes 2 & 3) or without (right panel lanes 4 & 5) HiFi-Taq ligase. Reaction products were then treated with T5 exonuclease at 37°C. for one hour and analyzed on a gel containing ethidium bromide. The yellow box highlights exonuclease-resistant circular DNA products generated in a CHTLA reaction with HiFi-Taq ligase (lane 3) but not in a control CHTLA reaction without ligase (lane 5). Lane 1, DNA ladder.

FIG. 4. CHTLA synthesis of ^{SCS}DNA from linear precursors. Plasmid-derived DNA precursors (restriction fragments, white arrow lanes 1 & 4) were ligated in a 10-cycle CHTLA reaction. CHTLA converted precursor DNA into relaxed circular (Lane 2, blue arrow) and supercoiled (Lane 2, yellow arrow) DNA. Precursors (Lanes 4-7) or CHTLA products (Lanes 8-10) were treated with T5 exonuclease (units shown in blue box). T5 treatment completely eliminated linear DNA precursors (Lanes 5-7), but ^{SCS}DNA CHTLA products were T5 exonuclease resistant resistant (Lanes 9-11).

FIG. 5A. ^{SCS}DNA generated by CHTLA is negatively supercoiled. To prepare CHTLA precursors to generate ^{SCS}DNA, plasmid pBluescript —SK (−), was digested with either BamHI or KpnI. The digested DNA was purified using a mini-spin column to remove the restriction enzymes. The purified DNA was mixed at a 1:1 ratio, then ligated in 10-cycle CHTLA reaction using HiFi-Taq DNA ligase. T5 exonuclease was used to remove unreacted precursors.

FIG. 5B. The ^{SCS}DNA was analyzed using agarose gel electrophoresis analysis without chloroquine or with 2.5 ug/ml chloroquine in the gel. The gel was stained with ethidium bromide after electrophoresis was complete. The yellow box indicates the location of ^{SCS}DNA with varying degrees of negative superhelical twist. The green arrow in the without chloroquine side indicates location of highly negatively supercoiled bacterially-sourced plasmid DNA. The red arrow indicates the shifted location of ^{SCS}DNA upon intercalation of chloroquine, which has collapsed into a single species. The green box indicates location of bacterially sourced plasmid DNA upon intercalation of chloroquine. Note that multiple species are now present indicating that the negative supercoiling has undergone varying degrees of relaxation, resulting in a series of bands with varying degrees of superhelical twisting. Pr, linearized precursor DNAs; VL, ^{SCS}DNA produced by CHTLA; PL, pBluescript —SK (−) purified from bacteria; L, 1 kb DNA ladder; EB, ethidium bromide, post-run, after electrophoresis.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,”the embodiments or elements presented herein, whether explicitly set forth or not.

As used herein, the term “heteroduplex” DNA molecule refers to a double-stranded molecule wherein the a first strand originates from one double-stranded or single stranded precursor DNA molecule, and a second strand originates from a different double-stranded or single-stranded DNA molecule, wherein the first strand and the second strand have been joined by Watson-Crick base pairing in the process known as annealing of, or hybridization of, complementary DNA strands (i.e., the first and second strands).

As used herein, the term “ligase” and “ligation agent” are used interchangeably and refer to any number of enzymatic or non-enzymatic reagents capable of joining DNA molecules, e.g., between two or more adjacent heteroduplexes with annealed compatible single-stranded ends, or within a single heteroduplex molecule with compatible single-stranded ends such that a circular molecule is formed through the establishment of new bonds. In some embodiments, a ligase is an enzymatic ligation reagent that, under appropriate conditions, forms phosphodiester bonds between the 3′-OH and the 5′-phosphate of adjacent nucleotides in DNA molecules, RNA molecules, oligonucleotides, or hybrids. Temperature sensitive ligases include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase, HiFi Taq Ligase, or Ampilgase. The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits.

As used herein, the term “overlapping sequence” refers to a sequence that is complementary in two polynucleotides, wherein a first polynucleotide comprises an overlapping sequence that is single-stranded (ss) and can be hybridized to a second polynucleotide comprising the complementary ss sequence.

As used herein, the term “overhang” refers to the single stranded region of double-stranded (ds) DNA at the end thereof and is either of type 5′or 3′due to the inherent directionality of DNA. The overhangs are generally generated in various lengths by treating double stranded DNA with restriction enzymes or exonucleases and/or by the addition of appropriate dNTPs (e.g., dATP, dTTP, dCTP, dGTP) through the action of an enzyme, i.e., terminal deoxynucleotidyl transferase. In some embodiments, the overhangs are in a range from 2 to 1000s of base pairs in length.

As used herein, the term “double stranded DNA” or “dsDNA” refers to oligonucleotides or polynucleotides having 3′ overhang(s), 5′ overhang(s) and/or blunt end(s) and comprise two single strands, all or part of which are complementary to each other, and thus dsDNA may contain a single stranded region at one or both ends and may be synthetic or natural origin derived from cells or tissues. In one embodiment, dsDNA is a product of PCR (Polymerase Chain Reaction) or fragments generated from genomic DNA or plasmids or vectors by a physical or enzyme treatment thereof.

As used herein, the term “buffering agent” refers to an agent that allows a solution to resist changes in pH when acid or alkali is added to the solution. Examples of suitable non-naturally occurring buffering agents that may be used in the compositions, kits, and methods described herein include, HEPES (4-(2-hydroxyethyl)-1-piperazineethanesulfonic acid), TAPS (tris(hydroxymethyl)methylamino]propanesulfonic acid), tricine (N-(2-Hydroxy-1,1-bis(hydroxymethyl)ethyl)glycine), phosphates, citrates, ammonium, acetates, carbonates, tris(hydroxymethyl)aminomethane (TRIS), TRIS-HCl, 3-(N-morpholino) propanesulfonic acid (MOPS), 3-morpholino-2-hydroxypropanesulfonic acid (MOPSO), 2-(N-morpholino)ethanesulfonic acid (MES), N-(2-Acetamido)-iminodiacetic acid (ADA), piperazine-N, N′-bis(2-ethanesulfonic acid) (PIPES), N-(2-Acetamido)-2-aminoethanesulfonic acid (ACES), cholamine chloride, N, N-Bis(2-hydroxyethyl)-2-aminoethanesulfonic acid (BES), 2-[[1,3-dihydroxy-2-(hydroxymethyl)propan-2-yl]amino]ethanesulfonic acid (TES), acetamidoglycine, glycinamide, and bicine (2-(Bis(2-hydroxyethyl)amino)acetic acid) buffers. It should be appreciated that the buffering agents can further at least one additional species such as: electrolytes such as MgCl₂, NaCl, and KCl; metal ions; type II topoisomerases (e.g., DNA gyrase); single-stranded DNA binding protein or thermostable single-stranded binding protein; crowding agents (e.g. polyethylene glycol); a redox reagent such as dithiothreitol (DTT); nicotinamide adenine dinucleotide (NAD); detergents; and non-ionic surfactants such as TRITON™ X-100 (Octylphenol Decaethylene Glycol Ether).

As used herein, the terms “DNA” or “RNA” is defined as a “polynucleotide” and may encompass primers, oligonucleotides, nucleic acid strands, etc. The DNA or RNA may be single stranded or double stranded or an admixture thereof. Such DNA or RNA polynucleotides may be synthetic, for example, synthesized in a DNA synthesizer, or naturally occurring, for example, extracted from a natural source, or derived from cloned or amplified material. Polynucleotides referred to herein may contain modified bases. Additionally, the DNA or RNA sequences may comprise one or more random or variable nucleotides. The use of randomized nucleotides may also include sequence-restricted regions, wherein sequence restricted means limiting the variation at one position to 2 or 3 nucleotide choices (i.e., A or C; A, G, or C etc.) rather than all 4 (ATGC). Typically, a polynucleotide contains a 5′ phosphate at one terminus (“5′ terminus”) and a 3′ hydroxyl group at the other terminus (“3′ terminus”) of the chain.

The nucleic acids utilized herein can be any nucleic acid, for example, human nucleic acids, bacterial nucleic acids, or viral nucleic acids. The nucleic acid sample can be, for example, a nucleic acid sample from one or more cells, tissues, or bodily fluids such as blood, urine, semen, lymphatic fluid, cerebrospinal fluid, or amniotic fluid, or other biological samples, such as tissue culture cells, buccal swabs, mouthwashes, stool, tissues slices, biopsy aspiration, and archeological samples such as bone or mummified tissue. Nucleic acids can be, for example, DNA, RNA, or the DNA product of RNA subjected to reverse transcription. Nucleic acids can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwashes, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources. In some embodiments, the nucleic acids do not comprise bacterial or viral nucleic acids.

As used herein, “Taq ligase”and “thermostable ligase”are synonymous.

As used herein, “synthetic circular supercoiled DNA” or ^{SCS}DNA is intended to capture products consisting of only synthetic circular DNA as well as products comprising some portion of synthetic circular DNA and synthetic supercoiled DNA, wherein the DNA precursors that give rise to the ^{SCS}DNA may be derived in part or wholly from a natural source (e.g., plasmid). In some embodiments, the synthetic supercoiled DNA is substantially supercoiled.

As used herein, synthetic circular supercoiled DNA and covalently closed circular DNAs are understood as being substantially exonuclease resistant. As used herein, any exonucleases are understood to lack or have minimal endonuclease activity using double-stranded DNA as a substrate.

As defined herein, “supercoil” or “supercoiling” is understood to be a global contortion of circular DNA. Supercoiling is understood to be the sum of the “twist” and the “writhe,” wherein the twist is the number of helical turns in the DNA and the writhe is the number of times the double helix crosses over itself. Positive and negative supercoiling is understood by the skilled artisan, but briefly positive supercoiling comprises extra, or additional, helical twists (i.e., is over wound) relative to the relaxed state while negative supercoiling comprises less, or subtractive, helical twists (i.e., is under wound) relative to the relaxed state. The DNA of most organisms is negatively supercoiled although it is understood that some amount of positive supercoiling is also present. Negative supercoiling advantageously allows processes such as transcription, DNA replication and recombination. Another term common to supercoiling is the “linking number,” or “Lk” which is understood to be the sum of the twist (T) and the writhe (W). The linking number is useful because it is a metric to identify changes of topology of DNA, e.g., as a result of enzymatic breakage and rejoining events. Of interest is the ΔLk, which is determined by the formula ΔLk=Lk-Lkₘ, wherein Lkₘ is the linking number for a relaxed circular DNA and Lk is the linking number for the circular DNA that has undergone supercoiling. Notably, Lk and Lkₘ are rounded to the nearest whole integer prior to the calculation of ΔLk. For a circular DNA undergoing negative supercoiling, the ΔLk will be negative.

As defined herein, “precursor” DNA fragments include dsDNA molecules (i.e., PCR products, restriction enzyme fragments, chemically or enzymatically produced DNA), single-stranded DNA molecules, DNA oligonucleotides, or mixtures thereof. Nucleotide bases within said precursor DNA fragments can be native adenine, guanine, cytosine, thymine, or any chemically modified form thereof that can be incorporated into a DNA molecule by chemical synthesis or the action of an enzyme, i.e., a DNA polymerase, or that can be chemically or enzymatically caused to appear in a base or bases after synthesis. The precursor DNAs can range from about 20 nucleotides to thousands to millions of nucleotides in length, and more preferably from about 200 to 10000 nucleotides for double-stranded precursors, and 30 to 200 for single-stranded precursors. In some embodiments, substantially “perfect” blunt ended DNA precursors are obtained for use by employing a Type IIS restriction enzyme (i.e., MlyI) to “polish” the ends of the precursor DNA fragments prior its use in a HTLA or CHTLA reaction, as understood by the person of skill in the art. In some embodiments, the precursor DNA fragments are generated using the methods described in U.S. Provisional Ser. No. 63/554,752 filed on Feb. 16, 2024 in the name of Xiang LI and Charles J. BIEBERICH and entitled “Method for generating circular DNA using heteroduplex thermostable ligation assembly of precursors created by rolling circle amplification,”which is hereby incorporated by reference herein in its entirety.

It is well known to the person skilled in the art that each nucleotide in a dsDNA molecule will pair with its Watson-Crick counterpart, also known as the “complementary” nucleotide. Further, it is understood that dsDNA sequences are represented by an upper or first (sense) strand sequence going in the direction from its 5′-to 3′-end and thus the complementary sequence is the sequence of the lower or second (antisense) strand in the same direction as the upper strand. When DNA sequences are stated as complementary, it is understood that when they anneal or hybridize, a double stranded DNA with antiparallel strands form. It is further understood that annealed complementary DNA sequences may include one or more non-canonical (i.e., Watson-Crick) base pairs or modified nucleotides that pair with multiple other nucleotides (e.g., Deoxyinosine may also pair with the three other DNA bases (deoxythymidine (dT), dA and dG).

As introduced hereinabove, covalently closed circular DNA that are completely devoid of bacterial DNA (e.g. minicricles) have recently emerged as a powerful gene delivery platform, but GMP production is highly problematic. The present inventors previously developed an improved DNA assembly process termed Heteroduplex Thermostable Ligase Assembly (HTLA™) which is capable of building and commercializing large linear DNA molecules completely in vitro, as described in International Patent Application No. PCT/US2023/064977 filed on Mar. 27, 2023 in the name of Charles J. Bieberich and Xiang Li, and entitled “HETERODUPLEX THEROMSTABLE LIGATION ASSEMBLY (HTLA) AND/OR CYCLIC HETERODUPLEX THERMOSTABLE LIGATION ASSEMBLY (CHTLA) FOR GENERATING DOUBLE-STRANDED DNA FRAGMENTS WITH SINGLE-STRANDED STICKY ENDS,” which is hereby incorporated herein in its entirety. HTLA is a straightforward assembly platform that generates ligation-ready single-stranded overhangs of user-defined length to create sticky-end blocks (SEBs) for assembly into higher order linear or circular structures. The starting material for HTLA or CHTLA is dsDNA or oligonucleotide precursors that self-assemble precisely to create much longer DNAs. As recited in International Patent Application No. PCT/US2023/064977, at least three precursors were used as reactants.

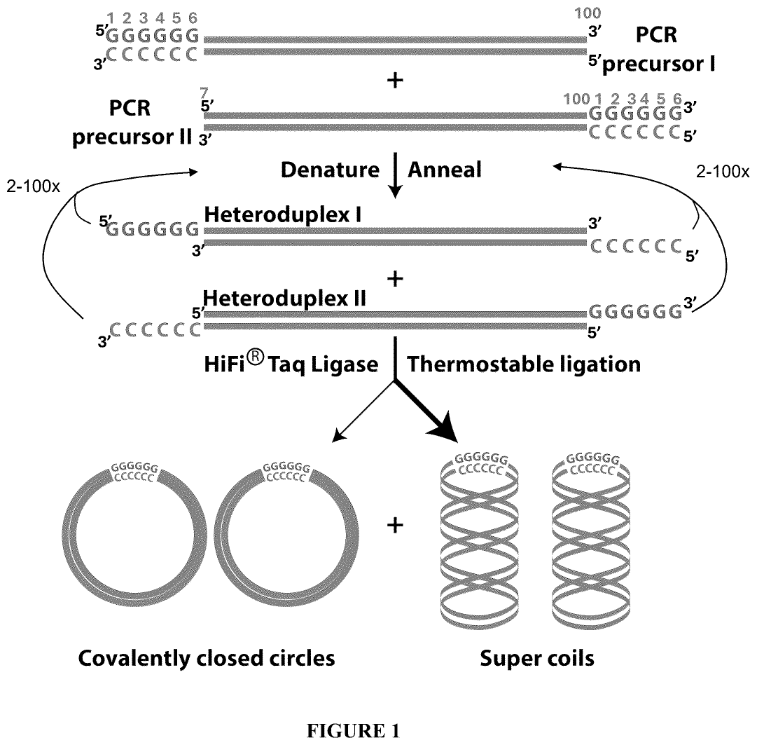

Briefly, the HTLA process is an efficient DNA assembly process that generates ligation-ready, user-defined, heteroduplex DNAs having lengths from one to thousands (or more) of nucleotides that comprise 5′ or 3′ single-stranded overhangs, or “sticky-ends,” that can join to form closed circular DNA molecules from double-stranded or single-stranded DNA precursor molecules (see, e.g., FIG. 1). When such process is performed for one cycle, it is referred to as HTLA while for more than one cycle, the process is referred to as Cyclic Heteroduplex Thermostable Ligase Assembly (CHTLA). As recited in International Patent Application No. PCT/US2023/064977, at least three precursors were used as reactants.

Surprisingly, in addition to making linear DNA, HLTA and CHTLA can also be used to generate closed circular DNA starting with as few as two precursors, e.g., dsDNA precursors or readily available and inexpensive DNA oligonucleotides (oligos). Further, it was unexpectedly discovered that circular and supercoiled DNA can be efficiently obtained without using precursors that comprise intentional nicks and without the addition of bending protein (e.g., Abf2p or HMGB1). As described herein, the methods comprise providing at least two user-designed DNA precursors to create a DNA sequence wherein a portion of a first strand and a second strand of the DNA sequence overlap with each other, and simultaneously complementary ‘sticky’ ends are generated that can be complimentary to one another and be ligated to form a covalently closed circle. This procedure is shown in FIG. 1. The dsDNA (or oligo) precursor mixture is heated and cooled in a buffer, heteroduplex molecules I and II form, and a DNA ligase joins the heteroduplex molecules to form covalently closed circular, plasmid vector-less, DNA molecules (synthetic circular supercoiled DNA, ^{SCS}DNA). In some embodiments, performing multiple rounds of heating and cooling (i.e., CHTLA) increases ^{SCS}DNA yield. As noted, the methods include the formation of intermediary heteroduplexes having sticky-end blocks (SEB), wherein a portion of the heteroduplexes have 5′ overhangs and a portion of the heteroduplexes have 3′ overhangs. In some embodiments, the 5′ overhang of a heteroduplex on one strand is complimentary to the 5′ overhang on the other strand of the same heteroduplex such that the heteroduplex circularizes (i.e., closes upon itself) upon ligation of the 5′ end of each strand with its own 3′ end. In some embodiments, the 3′ overhang of a heteroduplex on one strand is complimentary to the 3′ overhang on the other strand of the same heteroduplex such that the heteroduplex circularizes (i.e., closes upon itself) upon ligation of the 5′ end of each strand with its own 3′ end. In some embodiments, the circular and supercoiled DNA described herein are produced without plasmid vectors, and thus are devoid of bacterial DNA.

Even more unexpected was the formation of supercoiled DNA using the HTLA and CHTLA processes because DNA ligases are not known to have topoisomerase activity. DNA supercoiling is known to be important for DNA packaging within all cells. Without supercoiling, which reduces the space of the DNA, it would not be possible to package DNA into the cell. Furthermore, transfection of supercoiled DNA into cells (i.e. to genetically modify the cells for some purpose) is substantially more efficient than transfection of the same DNA in its linear or open (relaxed) circular form. In one embodiment, following denaturing, annealing, and ligation, some of the ^{SCS}DNA produced undergoes negative supercoiling in the presence of a DNA ligase. In some embodiments, a type II topoisomerase can be introduced to the ^{SCS}DNA (e.g., by having a thermostable type II topoisomerase in the milieu where the ^{SCS}DNA has formed or by adding type II topoisomerase to the milieu where the ^{SCS}DNA has formed at the appropriate time and at the appropriate temperature) to substantially supercoil the ^{SCS}DNA.

For the purposes of the present application, any enzyme that behaves like a type II topoisomerase that can induce DNA supercoiling is permissible. In some embodiments, the type II topoisomerase is a DNA gyrase or Topoisomerase IV. It was previously reported that DNA gyrase works via a transient double-strand break in the DNA, rather than nicks, and that DNA gyrase changes the linking number of DNA in steps of two (P. O. Brown and N. R. Cozzarelli, Scince, 1979, 206(4422), 1081-1083). As discussed herein, the presence of a type II topoisomerase such as DNA gyrase substantially supercoils the ^{SCS}DNA, relative to any supercoiling that may occur to the ^{SCS}DNA during a CHTLA reaction in the absence of type II topoisomerase. Accordingly, for the purposes of the present application, “substantially supercoiled” or “substantial supercoiling” corresponds to a degree of supercoiling that is induced by type II topoisomerase that is greater than supercoiling in ^{SCS}DNA produced during a CHTLA reaction in the absence of type II topoisomerase. Relating this to the linking number, for the purposes of the present application, ΔLk_{SS}=Lk_{SS}−Lk_{X} can be calculated, wherein the Lk_{SS} is the linking number of the ^{SCS}DNA substantially supercoiled in the presence of type II topoisomerase and Lk is the linking number of the ^{SCS}DNA in the absence of the type II topoisomerase, wherein a calculated ΔLk_{SS} (e.g., −2, −4, −6, −8, −10, etc), corresponds to substantial supercoiling.

It should be appreciated by the person skilled in the art that some type II topoisomerases are thermostable at temperatures of at least about 95° C. , while others are not. In some embodiments, the thermostable type II topoisomerase species is thermostable at temperatures in a range from about 37° C. to 100° C., or about 60° C. to 100° C., or about 80° C. to 100° C., for time in a range from about 30 seconds to about 10 minutes, or about 1 minute to about 5 minutes. Accordingly, when “thermostable type II topoisomerase” is used hereinafter, it is understood that the type II topoisomerase can survive the higher temperature of the denaturing/melting process, while maintaining some or all of its activity. In some embodiments, the thermostable type II topoisomerase is a thermostable DNA gyrase. Thermostable DNA gyrase is known in the art and can be engineered to be thermostable over the preferred temperature range.

In a first aspect, a method of forming Sticky-End Blocks (SEB) with 5′ or 3′ overhangs is described, the method comprising:
introducing at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature; and
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having 3′ overhangs.

In some embodiments, the method of forming SEB is a one-pot method. In some embodiments, the second temperature is lower than the first temperature. In some embodiments, the at least two precursor DNA fragments are selected from dsDNA molecules, single-stranded DNA molecules, DNA oligonucleotides, or mixtures thereof. In some embodiments, there are only two precursor DNA fragments that following denaturing, annealing, and ligation, forming two heteroduplex species, e.g., as shown in FIG. 1.

In some embodiments, the desired SEB products of the first aspect may be purified, for example, by agarose gel purification, or any other means of separation, from precursor DNA fragments, for subsequent ^{SCS}DNA and/or supercoil production in a second and/or third reaction.

In some embodiments, the buffer medium for the first aspect comprises a buffer to maintain pH. In some embodiments, the buffer comprises a combination of ATP, Tris-HCl, MgCl₂, KCl, NaCl, beta-mercaptoethanol, DTT, NAD, ATP, and Triton™ X-100, to maintain a pH of about 4 to about 12, or about 6 to 10, or about 7.5 to about 9. The denaturing (also referred to as “melting” or the first) temperature can be in a range from about 37° C. to 100° C., or about 60° C. to 100° C., or about 80° C. to 100° C., for time in a range from about 0.1 minutes to about 60 minutes, or about 1 minute to about 5 minutes. The annealing is conducted by lowering the temperature (i.e., the second temperature) about 5° C. to about 60° C. lower than the denaturing temperature, or lowering about 10° C. to 40° C. lower than the denaturing temperature, for time in a range from about 0.1 minutes to about 60 minutes, or about 4 minutes to 6 minutes.

In some embodiments, the precursor DNA fragments of FIG. 1 and all precursors described herein are specifically user-defined and user-designed. For example, consider the simple case for the design of two double stranded precursors (I and II) needed to create a 100 base-pair SEB heteroduplex I, wherein 94 base pairs overlap and are double stranded, and both ends have a 5′ overhang. In this example, the 5′ overhangs (i.e., GGGGGG and CCCCCC) are complementary. The converse heteroduplex II with complementary 3′ overhangs is also formed. Precursor I could be a double-stranded molecule comprised of base pairs 1 to 100. Precursor II could be a double stranded molecule comprised of base pairs 7 to 100 followed by 1-6 (see, e.g., green 1-6 in Precursor II). In this example, a first strand of nucleotides 1-100 of Precursor I and a second strand of nucleotides 7 to 100 followed by 1-6 of Precursor II are complimentary to one another in bases 7-100. Upon denaturing and annealing to form heteroduplex I, the first strand of nucleotides 7-100 of Precursor I and the second strand of nucleotides 7 to 100 followed by 1-6 of Precursor II anneal, yielding a 94 base-pair double-stranded region. Since nucleotides 1-6 (see, e.g., green 1-6 in Precursor I) of Precursor I do not have complementary bases in Precursor II, they remain single stranded. Likewise, nucleotides 1-6 in Precursor II do not have complementary bases in Precursor I, so they remain single stranded. That said, if the precursors were designed such that the 5′ overhangs of heteroduplex I are complimentary, they will join and form 100 base-pair ^{SCS}DNA during ligation. The same is true of Heteroduplex II which is comprised by 3′ overhangs. It is axiomatic that, in addition to the formation of SEB products, the two Precursors I and II can also reform by the re-annealing of their complementary strands. It should be understood by the person skilled in the art that the overhangs can be more or less than six base pairs represented in this example, for example from 2 to 1000s of base pairs, and can comprise any combination of nucleotides, so long as they are complementary to one another so that a covalently closed circle can be formed upon ligation of the complementary overhangs.

In a second aspect, a method of producing synthetic circular supercoiled DNA (^{SCS}DNA) is described, the method comprising:
introducing at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature; and
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced.

In some embodiments, the method of producing synthetic circular supercoiled DNA is a one-pot method. In some embodiments, the second temperature is lower than the first temperature. In some embodiments, the application of heat to a first temperature to denature and the lowering to a second temperature to anneal is repeatedly performed in cycles. In some embodiments, the number of cycles is in a range from 1 cycle to 100 cycles. In some embodiments, the at least two precursor DNA fragments are selected from dsDNA molecules, single-stranded DNA molecules, DNA oligonucleotides, or mixtures thereof. In some embodiments, there are only two precursor DNA fragments that following denaturing, annealing, and ligation, forming two heteroduplex species, e.g., as shown in FIG. 1. In some embodiments, a portion of the ^{SCS}DNA undergoes negative supercoiling in the presence of the thermostable DNA ligase enzyme. In some embodiments, a single stranded DNA binding protein or thermostable single stranded DNA binding protein is present in the reaction, e.g., added to the buffer medium or added to the milieu at some point prior to annealing, to facilitate annealing of DNA strands.

In some embodiments of the second aspect, the buffer medium comprising a thermostable DNA ligase enzyme comprises a buffer to maintain pH. In some embodiments, the buffer comprises a combination of ATP, Tris-HCl, MgCl₂, KCl, NaCl, beta-mercaptoethanol, DTT, NAD, and Triton™ X-100, to maintain a pH of about 4 to about 12, or about 6 to 10, or about 7.5 to about 9. The denaturing (also referred to as “melting” or the first) temperature can be in a range from about 37° C. to 100° C., or about 60° C. to 100° C., or about 80° C. to 100° C., for time in a range from about 0.1 minutes to about 60 minutes, or about 1 minute to about 5 minutes. The annealing is conducted by lowering the temperature (i.e., the second temperature) about 5° C. to about 60° C. lower than the denaturing temperature, or lowering about 10° C. to 40° C. lower than the denaturing temperature, for time in a range from about 0.1 minutes to about 60 minutes, or about 4 minutes to 6 minutes. Accordingly, in some embodiments, the second temperature is in a range from about 25° C. to about 85° C., or about 25° C. to about 70° C., or about 25° C. to about 65° C., or about 37° C. to about 65° C., or 50° C. to about 70° C., for time in a range from about 0.1 minutes to about 60 minutes, or about 4 minutes to 6 minutes. As noted, the denaturing/annealing process can be repeatedly cycled about 2 to about 100 times, wherein each cycle increases the yield of ^{SCS}DNA. Accordingly, in some embodiments, the nucleic acid ligation schemes are temperature cycling from, e.g., about 80° C. to 100° C., to a lower temperature of about 40° C. to about 70°C, for 2 to 100 cycles.

In some embodiments, the method of producing synthetic circular supercoiled DNA (^{SCS}DNA) comprises: introducing at least two precursor DNA fragments into a buffer medium at a pH of about 7.5 to about 9 and comprising ATP, Tris-HCl, MgCl₂, KCl, NaCl, beta-mercaptoethanol, NAD, DTT, Triton X-100, and at least one thermostable ligase, wherein the precursor DNA fragments will correctly assemble to generate a defined DNA sequence;
applying heat at a first temperature to cause the at least two precursor DNA fragments to denature, wherein the first temperature is determined by the size of the DNA sequence and can range from about 37° C. to 100° C. for time in a range from about 0.1 minutes to about 60 minutes ; and
lowering the temperature to a second temperature for annealing in the presence of the thermostable DNA ligase enzyme, wherein the second temperature is from 10° C. to 40° C. lower than the first temperature for time in a range from about 4 minutes to about 10 minutes, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced.

In some embodiments, the application of heat to a first temperature to denature and the lowering to a second temperature to anneal is repeatedly performed in cycles. In some embodiments, the number of cycles is in a range from 2 cycles to 100 cycles. In some embodiments, the at least two precursor DNA fragments are selected from dsDNA molecules, single-stranded DNA molecules, DNA oligonucleotides, or mixtures thereof. In some embodiments, there are only two precursor DNA fragments that following denaturing, annealing, and ligation, forming two heteroduplex species, e.g., as shown in FIG. 1. In some embodiments, a portion of the ^{SCS}DNA undergoes negative supercoiling in the presence of the thermostable DNA ligase enzyme. In some embodiments, a single stranded DNA binding protein or thermostable single stranded DNA binding protein is present in the reaction, e.g., added to the buffer medium or added to the milieu at some point prior to annealing, to facilitate annealing of DNA strands.

In a third aspect, a method of producing substantially supercoiled DNA is described, the method comprising:
introducing at least two precursor DNA fragments into a buffer medium comprising a thermostable DNA ligase enzyme and a thermostable type II topoisomerase, wherein the precursor DNA fragments will correctly assemble to generate a defined DNA sequence;
applying heat to a first temperature to cause the at least two precursor DNA fragments to denature;
lowering the temperature to a second temperature for: (i) annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and (ii) substantial supercoiling of the ^{SCS}DNA in the presence of the thermostable type II topoisomerase.

In some embodiments, the method of producing substantially supercoiled DNA is a one-pot method. In some embodiments, the second temperature is lower than the first temperature. In some embodiments, the application of heat to a first temperature to denature and the lowering to a second temperature to anneal/ligase/supercoil is repeatedly performed in cycles. In some embodiments, the number of cycles is in a range from 2 cycles to 100 cycles. In some embodiments, the at least two precursor DNA fragments are selected from dsDNA molecules, single-stranded DNA molecules, DNA oligonucleotides, or mixtures thereof. In some embodiments, the thermostable type II topoisomerase comprises DNA gyrase or Topoisomerase IV. In some embodiments, there are only two precursor DNA fragments that following denaturing, annealing, and ligation, forming two heteroduplex species, e.g., as shown in FIG. 1. In some embodiments, there are 3, 4, 5, 6, 7, 8, 9, or 10 or more precursor DNA fragments, as understood by the person skilled in the art. In some embodiments, a single stranded DNA binding protein or thermostable single stranded DNA binding protein is present in the reaction, e.g., added to the buffer medium or added to the milieu at some point prior to annealing, to facilitate annealing of DNA strands.

In some embodiments of the third aspect, the buffer medium comprising a thermostable DNA ligase enzyme and a thermostable type II topoisomerase comprises a buffer to maintain pH. In some embodiments, the buffer comprises a combination of ATP, Tris-HCl, MgCl₂, KCl, NaCl, beta-mercaptoethanol, DTT, NAD, and Triton™ X-100, to maintain a pH of about 4 to about 12, or about 6 to 10, or about 7.5 to about 9. The denaturing (also referred to as “melting” or the first) temperature can be in a range from about 37° C. to 100° C., or about 60° C. to 100° C., or about 80° C. to 100° C., for time in a range from about 0.1 minutes to about 60 minutes, or about 1 minute to about 5 minutes. The annealing/ligation/supercoiling is conducted by lowering the temperature (i.e., the second temperature) about 5° C. to about 60° C. lower than the denaturing temperature, or lowering about 10° C. to 40° C. lower than the denaturing temperature, for time in a range from about 0.1 minutes to about 60 minutes, or about 4 minutes to 6 minutes. Accordingly, in some embodiments, the second temperature is in a range from about 25° C. to about 85° C., or about 25° C. to about 70° C., or about 25° C. to about 65° C., or about 37° C. to about 65° C., or 50° C. to about 70° C., for time in a range from about 0.1 minutes to about 60 minutes, or about 4 minutes to 6 minutes. As noted, the process or raising the temperature to the first temperature and lowering to the second temperature can be repeatedly cycled about 2 to about 100 times, wherein each cycle increases the yield of supercoiled DNA molecules and the extent of supercoiling of individual molecules. Accordingly, in some embodiments of the third aspect, the supercoiling schemes are temperature cycling from, e.g., a first temperature of about 80° C. to 100° C. to a second temperature of about 40° C. to about 70°C, for 2to 100 cycles.

In some embodiments, the method of producing substantially supercoiled DNA comprises:
introducing at least two precursor DNA fragments into a buffer medium at a pH of about 7.5 to about 9 and comprising ATP, Tris-HCl, MgCl₂, KCl, NaCl, beta-mercaptoethanol, NAD, DTT, Triton X-100, at least one thermostable ligase and a thermostable type II topoisomerase, wherein the precursor DNA fragments will correctly assemble to generate a defined DNA sequence;
applying heat at a first temperature to cause the at least two precursor DNA fragments to denature, wherein the first temperature for denaturing is determined by the size of the DNA sequence and can range from about 37° C. to 100° C. for time in a range from about 0.1 minutes to about 60 minutes; and
lowering the temperature to a second temperature for: (i) annealing in the presence of the thermostable DNA ligase enzyme, wherein the second temperature is from 10° C. to 40° C. lower than the first temperature for time in a range from about 4 minutes to about 10 minutes, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and (ii) substantial supercoiling of the ^{SCS}DNA in the presence of the thermostable type II topoisomerase.

In some embodiments, the method of producing substantially supercoiled DNA is a one-pot method. In some embodiments, the application of heat to a first temperature to denature and the lowering to a second temperature to anneal and produce ^{SCS}DNA and supercoils is repeatedly performed in cycles. In some embodiments, the number of cycles is in a range from 2 cycles to 100 cycles. In some embodiments, the at least two precursor DNA fragments are selected from dsDNA molecules, single-stranded DNA molecules, DNA oligonucleotides, or mixtures thereof. In some embodiments, the thermostable type II topoisomerase comprises DNA gyrase. In some embodiments, there are only two precursor DNA fragments that following denaturing, annealing, and ligation, forming two heteroduplex species, e.g., as shown in FIG. 1. In some embodiments, there are 3, 4, 5, 6, 7, 8, 9, or 10 or more precursor DNA fragments, as understood by the person skilled in the art. In some embodiments, a single stranded DNA binding protein or thermostable single stranded DNA binding protein is present in the reaction, e.g., added to the buffer medium or added to the milieu at some point prior to annealing, to facilitate annealing of DNA strands.

In a fourth aspect, a method of producing substantially supercoiled DNA is described, the method comprising:
introducing the at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature;
lowering the temperature to a second temperature for annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having single-stranded 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and
lowering the temperature to a third temperature and adding a bolus of type II topoisomerase to initiate substantial supercoiling of the ^{SCS}DNA in the presence of the type II topoisomerase.

In some embodiments, the second temperature is lower than the first temperature. In some embodiments, the third temperature is lower than the second temperature. In some embodiments, the application of heat to a first temperature to denature, the lowering to a second temperature to anneal, and the further lowering to a third temperature to supercoil is repeatedly performed in cycles. In some embodiments, the number of cycles is in a range from 2 cycles to 100 cycles. In some embodiments, the at least two precursor DNA fragments are selected from dsDNA molecules, single-stranded DNA molecules, DNA oligonucleotides, or mixtures thereof. In some embodiments, the type II topoisomerase comprises DNA gyrase or Topoisomerase IV. In some embodiments, there are only two precursor DNA fragments that following denaturing, annealing, and ligation, forming two heteroduplex species, e.g., as shown in FIG. 1. In some embodiments, there are 3, 4, 5, 6, 7, 8, 9, or 10 or more precursor DNA fragments, as understood by the person skilled in the art. In some embodiments, a single stranded DNA binding protein or thermostable single stranded DNA binding protein is present in the reaction, e.g., added to the buffer medium or added to the milieu at some point prior to annealing, to facilitate annealing of DNA strands.

In some embodiments of the third aspect, the buffer medium comprising a thermostable DNA ligase enzyme comprises a buffer to maintain pH. In some embodiments, the buffer comprises a combination of ATP, Tris-HCl, MgCl₂, KCl, NaCl, beta-mercaptoethanol, DTT, NAD, and Triton™ X-100, to maintain a pH of about 4 to about 12, or about 6 to 10, or about 7.5 to about 9. The denaturing (also referred to as “melting” or the first) temperature can be in a range from about 37° C. to 100° C., or about 60° C. to 100° C., or about 80° C. to 100° C., for time in a range from about 0.1 minutes to about 60 minutes, or about 1 minute to about 5 minutes. The annealing is conducted by lowering the temperature (i.e., the second temperature) about 5° C. to about 60° C. lower than the denaturing temperature, or lowering about 10° C. to 40° C. lower than the denaturing temperature, for time in a range from about 0.1 minutes to about 60 minutes, or about 4 minutes to 6 minutes. Accordingly, in some embodiments, the second temperature is in a range from about 25° C. to about 85° C., or about 25° C. to about 70° C., or about 25° C. to about 65° C., or about 37° C. to about 65° C., or 50° C. to about 70° C., for time in a range from about 0.1 minutes to about 60 minutes, or about 4 minutes to 6 minutes. It should be appreciated by the person skilled in the art that the second temperature is greater than the third temperature. The third temperature is dependent on the nature of the type II topoisomerase and is selected to ensure that the type II topoisomerase does not undergo heat degradation and is available to supercoil the ^{SCS}DNA. As noted, the denaturing/annealing/supercoiling process can be repeatedly cycles about 2 to about 100 times, wherein each cycle increases the yield of supercoiled DNA molecules and the extent of supercoiling of individual molecules. Accordingly, in some embodiments, the nucleic acid supercoiling schemes are temperature cycling from, e.g., a first temperature of about 80° C. to 100° C., to a second temperature of about 40° C. to about 70°C, to a third temperature that is lower than the second temperature, for 2 to 100 cycles.

Referring to FIG. 1, it is noted that heteroduplex I and II can and do ligate to one another to form concatemers. However, without being bound by theory, intramolecular (or self) ligation to circularize is substantially favored over intermolecular ligation to form concatemers.

In some embodiments, following the production of ^{SCS}DNA and/or supercoiled DNA, concatemers and generally tangled DNA, as well as precursors, can be removed by treatment with a 5′ to 3′ or 3′ to 5′ exonuclease, for example, T5 or T7 exonucleases. The exonucleases can be degraded by proteinase K digestion and the DNA products are precipitated from solution. Alternatively, column-based methods are known in the art to deproteinate the sample and purify the DNA, as understood by the person skilled in the art.

As discussed hereinabove, in some embodiments, the 5′ overhang of one strand of a heteroduplex is complimentary to the 5′ overhang of the opposite strand of the same heteroduplex such that the heteroduplex circularizes (i.e., closes upon itself) upon annealing of the overhangs. Ligation of the 5′ end of one strand to the now juxtaposed 3′ end of the same strand results in a complete phosphodiester backbone on one strand. The same occurs on the opposite strand such that a covalently closed circular double stranded DNA results. Further, in some embodiments, the 3′ overhang of one strand of a heteroduplex is complimentary to the 3′ overhang of the opposite strand of the same heteroduplex such that the heteroduplex circularizes (i.e., closes upon itself) upon annealing of the overhangs. Ligation of the 3′ end of one strand to the now juxtaposed 5′ end of the same strand results in a complete phosphodiester backbone on one strand. The same occurs on the opposite strand such that a covalently closed circular double stranded DNA results.

In some embodiments, the circular and supercoiled DNA described herein are produced without plasmid vectors, and thus are devoid of bacterial DNA. Advantageously, because the DNA precursor fragments are user-designed, the DNA precursor fragment can be part of a designed vector (plasmid, cosmid, BAC, YAC, etc.) such that the precursor fragment is liberated from the vector (e.g. by restriction endonuclease digestion) and because sticky ends that are complementary to one another form in the heteroduplex molecules, then a complete closed circular vector can be obtained using the methods described herein, wherein the circular and supercoiled DNA product is devoid of bacterial DNA.

In some embodiments, the circular and supercoiled DNA described herein are produced with plasmid (or cosmid, BAC, YAC, etc.) vectors and a DNA sequence of interest, and thus have the structure of a conventional cloning vector plus insert but are generated completely synthetically through the process of HTLA or CHTLA.

Accordingly, in a fifth aspect, a synthetic circular supercoiled DNA (^{SCS}DNA) is described, wherein the ^{SCS}DNA is devoid of any DNA of bacterial or viral origin. In some embodiments, the ^{SCS}DNA is plasmid vector-less. In some embodiments, the ^{SCS}DNA of the fifth aspect is substantially supercoiled. The ^{SCS}DNA sequence can be completely user-defined (i.e. scarless) and generated in a one-pot method (e.g., any of the methods of the first, second, third or fourth aspect described herein) and produced in quantities useful for gene therapy, cell engineering (i.e. CAR-T therapy), vaccines, as well as genome engineering in bacteria, yeast or other organisms. For example, in some embodiments, the ^{SCS}DNA is a vaccine, and the precursor DNA fragments are user-designed to direct expression of a vaccine antigen, the precursor DNA fragments are produced in large quantities, and then the precursor DNA fragments undergo HTLA or CHTLA, as described herein, to produce ^{SCS}DNA that is plasmid vector-less and directs vaccine antigen expression. In another example, ^{SCS}DNA is used to carry a payload gene in a gene therapy setting, as naked DNA or in a formulation designed to enhance cellular uptake generally or in a cell or tissue-directed manner. In another example, the ^{SCS}DNA encodes one or more components required to produce lentiviruses, adeno-associated viruses, or other viruses containing specific payloads for gene therapy in living animals including humans or for modification of cells in culture. In another example, ^{SCS}DNA that has a structure that is identical to a conventional plasmid (or cosmid, BAC, YAC, etc.) but is produced completely synthetically, e.g. to produce a large quantity of low copy conventional vector with insert.

### EXAMPLES

### Example 1—^{SCS} Dna Produced by Chtla Is Partially Supercoiled

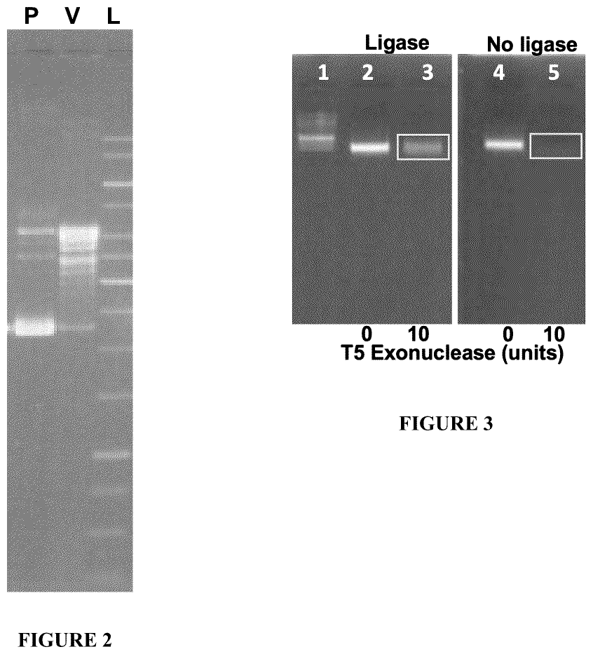

To demonstrate that CHTLA produces circular products with varying degrees of de novo supercoiling, plasmid pMaxGFP was digested with either KpnI or XhoI resulting in linear overlapping DNA fragments offset by 700 bp such that the offset regions were perfectly homologous. The digested DNA was purified to remove restriction enzymes and approximately five micrograms of each fragment was mixed (1:1) in a volume of 50 microliters of a buffer containing 20 mM Tris-HCl, 150 mM KCl, 10 mM MgCl₂, 10 mM dithiothreitol (DTT), 1 mM (nicotinamide adenine dinucleotide) NAD, 0.1% Triton™ X-100 (pH 8.5@25° C.) and 1 μL of 44 nM HiFi Taq ligase (New England Biolabs) and subjected to 10 cycles of heating and cooling from 95° C. to 60° C. in a standard thermocycler to generate ^{SCS}DNA. Upon completion of the 10-cycle CHTLA reaction, an aliquot of the products was treated with 10 units of T5 exonuclease at 37° C. for one hour. To demonstrate that the CHTLA products were supercoiled, the CHTLA products were analyzed on an agarose gel without ethidium bromide and were compared to plasmid pMaxGFP purified from bacteria. After electrophoresis the gel was stained with ethidium bromide. Analysis of the resulting gel revealed a series of bands in the lane containing T5 exonuclease-treated CHTLA products (see, FIG. 2, lane “V”). The bands correspond to closed circular CHTLA product DNA with varying degrees of superhelical twist, including a fraction that is supercoiled to the same maximal extent seen in the bacterially-derived plasmid pMaxGFP.

### Example 2—Chtla Synthesis of a Circular Dna Using Single-stranded Oligonucleotides As Precursors

To demonstrate that CHTLA converts single stranded oligonucleotides to closed circular products, six 5′-phophorylated 80-mer oligonucleotides overlapping by 40 nucleotides were subjected to CHTLA. The oligonucleotides were mixed at a final concentration of 2 micromolar each in in a volume of 50 microliters of a buffer containing 20 mM Tris-HCl, 150 mM KCl, 10 mM MgCl₂, 10 mM DTT, 1 mM NAD, 0.1% Triton™ X-100 (pH 8.5@25° C.) and 1 μL of 44 nM HiFi Taq ligase (New England Biolabs) and subjected to 10 cycles of heating and cooling from 95° C. to 60° C. in a standard thermocycler. A parallel, and identical, reaction was performed leaving out the HiFi Taq Ligase. Upon completion of the 10-cycle CHTLA reaction, an aliquot of the products were treated with 10 units of T5 exonuclease at 37° C. for one hour. CHTLA products with and without T5 exonuclease exposure were then analyzed on an agarose gel containing ethidium bromide in the gel. As seen in FIG. 3, a substantial portion of the CHTLA products were resistant to T5 exonuclease digestion, demonstrating that they are in a closed circular conformation.

### Example 3—Chtla Synthesis of Supercoiled ^{SCS}Dna From Linear Dsdna Precursors

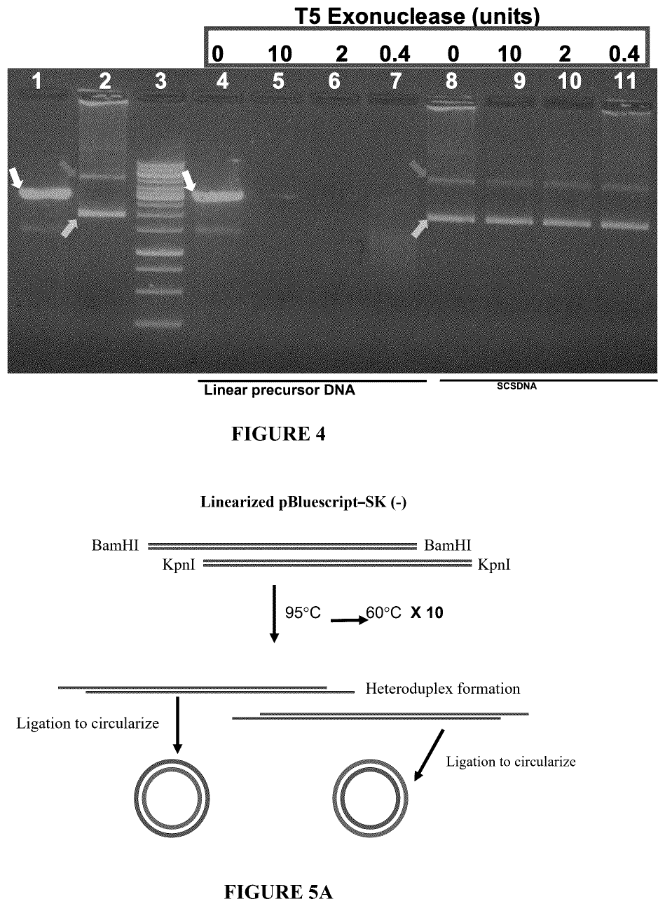

To demonstrate that CHTLA converts linear dsDNA precursors to closed circular and supercoiled circular products, plasmid pBluescript—SK (−) was digested with restriction enzymes BamHI and KpnI to yield 2 overlapping and linear DNA molecules with a 62 bp offset, such that the offset regions were perfectly homologous. Approximately five micrograms of each fragment were mixed (1:1) in a volume of 50 microliters of a buffer containing 20 mM Tris-HCl, 150 mM KCl, 10 mM MgCl₂, 10 mM DTT, 1 mM NAD, 0.1% Triton™ X-100 (pH 8.5@25° C.) and 1 μL of 44 nM HiFi Taq ligase (New England Biolabs) and subjected to 10 cycles of heating and cooling from 95° C. to 60° C. in a standard thermocycler. To demonstrate that the CHTLA products were almost entirely covalently closed circular DNA, the CHTLA products were treated with 10, 2, or 0.4 units of T5 exonuclease directly in the HiFi Taq ligase reaction buffer and incubated at 37° C. for one hour. Separate aliquots of linear CHLTA precursors were also treated with T5 exonuclease in parallel. The products of the T5 exonuclease reactions were then analyzed on an agarose gel containing ethidium bromide (in the gel), as shown in FIG. 4. Ethidium bromide intercalates into double stranded DNA and induces negative supercoiling of some but not all closed circular DNA during the process of electrophoretic separation. Analysis of the resulting gel image reveals that whereas all of the linear precursor molecules are completely digested by T5 exonuclease, approximately 90% of the CHTLA products are T5 exonuclease-digestion resistant, indicating that they are in a closed circular conformation. This data demonstrates that two-fragment HLTA using dsDNA precursors efficiently generates circular DNA molecules, and that a high percentage of the products are in the form of supercoiled ^{SCS}DNA. Furthermore, the closed circular CHTLA products become more supercoiled as a result of the intercalation of ethidium bromide during the process of electrophoresis.

### Example 4—^{SCS}Dna Generated by Chtla Is Negatively Supercoiled

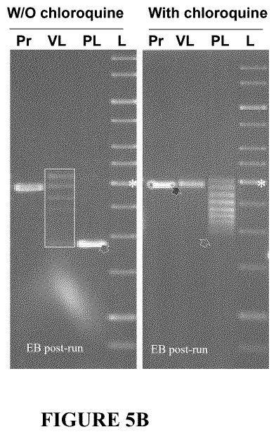

To demonstrate that ^{SCS}DNA generated by CHTLA is negatively supercoiled, ^{SCS}DNA, plasmid pBluescript—SK (−), was linearized with either BamHI or KpnI generating overlapping DNA molecules with a 62 bp offset (see, FIG. 5A). Approximately five micrograms of each fragment was mixed in a volume of 50 microliters of a buffer containing 20 mM Tris-HCl, 150 mM KCl, 10 mM MgCl₂, 10 mM DTT, 1 mM NAD, 0.1% Triton™ X-100 (pH 8.5@25° C.) and 1 μL of 44 nM HiFi Taq ligase (New England Biolabs) and subjected to 10 cycles of heating and cooling from 95° C. to 60° C. in a standard thermocycler. Upon completion of the 10-cycle CHTLA reaction, aliquots of the products were treated with 10 units of T5 exonuclease at 37° C. for one hour. The resulting T5 exonuclease-resistant products were resolved on agarose gels with or without chloroquine. Chloroquine intercalates into DNA and induces positive helical twisting. Neither gel contained ethidium bromide. After electrophoresis was complete, both gels were stained with ethidium bromide. In the gel without chloroquine, multiple bands representing circular ^{SCS}DNA with different degrees of superhelical twisting were observed (see, yellow box, column VL, FIG. 5B). With exposure to chloroquine, those multiple bands collapse “upwards” into a single species (see, red arrow, FIG. 5B). Likewise, bacterially sourced negatively supercoiled plasmid DNA also shifts upwards upon exposure to chloroquine (see, FIG. 5B, green arrow as move from side without chloroquine to side with chloroquine), and is observed as multiple species with varying degrees of superhelical twisting. Thus, both the ^{SCS}DNA and the bacterially sourced negatively supercoiled plasmid DNA shift in the same manner in the presence of chloroquine, demonstrating that ^{SCS}DNA is also negatively supercoiled, although to a lesser overall extent than bacterially sourced plasmid DNA.

What is claimed is:

1. A method of producing synthetic circular supercoiled DNA (^{SCS}DNA), the method comprising:
introducing at least two precursor DNA fragments into a buffer medium comprising a thermostable DNA ligase enzyme, wherein the precursor DNA fragments will correctly assemble to generate a defined DNA sequence;
applying heat to a first temperature to cause the at least two precursor DNA fragments to denature; and
lowering the temperature to a second temperature for: (i) annealing in the presence of the thermostable DNA ligase enzyme, thereby generating double-stranded DNA heteroduplexes formed by base pairing of complementary regions, a portion of the heteroduplexes having single-stranded 5′ overhangs and a portion of the heteroduplexes having 3′ overhangs, wherein when the 5′ intramolecular overhangs on a heteroduplex molecule are complementary and ligation on both DNA strands occurs, ^{SCS}DNA is produced, and when the 3′ intramolecular overhangs on a heteroduplex molecule are complementary and ligation on both DNA strands occurs, ^{SCS}DNA is produced; and (ii) substantial supercoiling of the ^{SCS}DNA in the presence of the thermostable type II topoisomerase.

2. The method of claim 1, wherein the method of producing ^{SCS}DNA is a one-pot method.

3. The method of claim 1, wherein the at least two precursor DNA fragments are selected from double stranded (ds) DNA molecules, single-stranded DNA molecules, DNA oligonucleotides, or mixtures thereof.

4. The method of claim 1, wherein two precursor DNA fragments are used.

5. The method of claim 1, wherein the first temperature is in a range from about 37° C. to about 100° C., and it is applied for time in a range from about 30 seconds to about 10 minutes.

6. The method of claim 1, wherein the second temperature is about 25° C. to about 85° C., and it is maintained for time in a range from about 0.1 minutes to about 60 minutes.

7. The method of claim 1, wherein the application of heat to a first temperature to denature and the lowering to a second temperature to anneal is repeatedly performed in cycles.

8. The method of claim 1, wherein during annealing, heteroduplex double-stranded DNA sequences are generated, formed by base pairing of complementary regions, wherein the heteroduplexes comprise 5′ or 3′ overhangs.

9. The method of claim 8, wherein ^{SCS}DNA is produced when the 5′ or 3′ overhangs are complementary.

10. The method of claim 1, wherein the buffer comprises a combination of ATP, Tris-HCl, MgCl₂, KCl, NaCl, DTT, beta-mercaptoethanol, NAD, and TRITON X-100.

11. The method of claim 10, wherein the pH is maintained at about 4 to about 12.

12. The method of claim 4, wherein a portion of the ^{SCS}DNA undergoes negative supercoiling in the presence of the thermostable DNA ligase enzyme.

13. The method of claim 10, wherein the buffer medium further comprises a type II topoisomerase.

14. The method of claim 13, wherein the ^{SCS}DNA are substantially supercoiled.

15. The method of claim 13, wherein the type II topoisomerase is thermostable and the thermostable type II isomerase can be cycled through the denaturing and annealing processes while maintaining some or all of its activity.

16. The method of claim 13, wherein the type II topoisomerase is thermostable or not thermostable and the method further comprises lowering the temperature to a third temperature and adding a bolus of type II topoisomerase to initiate substantial supercoiling of the ^{SCS}DNA in the presence of the type II topoisomerase.

17. The method of claim 13, wherein the type II topoisomerase is a DNA gyrase or Topoisomerase IV.

18. The method of claim 1, wherein the at least two precursor DNA fragments do not comprise intentional nicks and no bending proteins are added.

19. The method of claim 1, wherein the ^{SCS}DNA produced is devoid of bacterial DNA.

20. A synthetic circular supercoiled DNA (^{SCS}DNA) produced according to the method of claim 1.
